SEQUENCE LISTING (11 sequences)

SEQ ID NO: 1 - protein, 279 aa, Homo sapiens
WTYHYSTKAYSWNISRKYCQNRYTDLVAIQNKNEIDYLNKVLPYYSSYYWIGIRKNNKTWTWVGTKKALTNEAENWADNEPNNKRNNEDCVEIYIKSPSAPGKWNDEHCLKKKHALCYTASCQDMSCSKQGECLETIGNYTCSCYPGFYGPECEYVRECGELELPQHVLMNCSHPLGNFSFNSQCSFHCTDGYQVNGPSKLECLASGIWTNKPPQCLAAQCPPLKIPERGNMTCLHSAKAFQHQSSCSFSCEEGFALVGPEVVQCTASGVWTAPAPVCK

SEQ ID NO: 2 - protein, 15 aa, synthetic construct
  REGION 1..15: note = CDR1 light chain amino acid sequence
KASQSVDYDGHSYMN

SEQ ID NO: 3 - protein, 7 aa, synthetic construct
  REGION 1..7: note = CDR2 light chain amino acid sequence
AASNLES

SEQ ID NO: 4 - protein, 9 aa, synthetic construct
  REGION 1..9: note = CDR3 light chain amino acid sequence
QQSDENPLT

SEQ ID NO: 5 - protein, 112 aa, synthetic construct
  REGION 1..112: note = Mature light chain variable region amino acid sequence
DIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKR

SEQ ID NO: 6 - protein, 5 aa, synthetic construct
  REGION 1..5: note = CDR1 heavy chain amino acid sequence
SYDIN

SEQ ID NO: 7 - protein, 17 aa, synthetic construct
  REGION 1..17: note = CDR2 heavy chain amino acid sequence
WIYPGDGSIKYNEKFKG

SEQ ID NO: 8 - protein, 13 aa, synthetic construct
  REGION 1..13: note = CDR3 heavy chain amino acid sequence
RGEYGNYEGAMDY

SEQ ID NO: 9 - protein, 122 aa, synthetic construct
  REGION 1..122: note = Mature heavy chain variable region amino acid sequence
QVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSS

SEQ ID NO: 10 - protein, 218 aa, synthetic construct
  REGION 1..218: note = Light chain mature amino acid sequence
DIQMTQSPSSLSASVGDRVTITCKASQSVDYDGHSYMNWYQQKPGKAPKLLIYAASNLESGVPSRFSGSGSGTDFTLTISSLQPEDFATYYCQQSDENPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 448 aa, synthetic construct
  REGION 1..448: note = Heavy chain mature amino acid sequence
QVQLVQSGAEVKKPGASVKVSCKVSGYTFTSYDINWVRQAPGKGLEWMGWIYPGDGSIKYNEKFKGRVTMTVDKSTDTAYMELSSLRSEDTAVYYCARRGEYGNYEGAMDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCAVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

(19) United States
(12) Patent Application Publication
(10) Pub. No.: US 2024/0190971 A1
(43) Pub. Date: Jun. 13, 2024

(54) USE OF an anti-P-selectin antibody

(71) Applicants: Anna Rita Franco Migliaccio, Bologna (IT); NOVARTIS AG, Basel (CH)

(72) Inventors: Shalini CHATURVEDI, Princeton, NJ (US); Hans MENSSEN, Basel (CH); Anna Rita Franco Migliaccio, Bologna (IT); Thomas Radimerski, Basel (CH)

(21) Appl. No.: 18/330,844

(22) Filed: Jun. 7, 2023

Related U.S. Application Data
(63) Continuation of application No. 16/977,126, filed on Sep. 1, 2020, now abandoned.
(60) Provisional application No. 62/640,113, filed on Mar. 8, 2018.
(60) Provisional application No. 62/640,117, filed on Mar. 8, 2018.

(51) Int. Cl. C07K 16/28; A61K 31/519; A61K 39/00; A61K 39/395; A61P 35/00

(52) U.S. Cl. CPC C07K 16/2854; A61K 31/519; A61K 39/3955; A61P 35/00; A61K 2039/505; C07K 2317/24; C07K 2317/76

(57) ABSTRACT

The invention relates to the use of an anti-P-selectin antibody or binding fragment thereof, suitably crizanlizumab or a binding fragment thereof in the treatment of myelofibrosis (MF). The invention also relates to a pharmaceutical combination comprising a) an anti-P-Selectin antibody and b) at least one further therapeutic agent, preferably ruxolitinib or a pharmaceutically acceptable salt thereof.

The present invention relates to uses of an anti-P-selectin antibody and combinations thereof.

### FIELD OF THE INVENTION

The invention relates to the use of an anti-P-selectin antibody, or binding fragment thereof, in the treatment of myelofibrosis (MF). The invention also relates to a pharmaceutical combination comprising a) a P-Selectin binding antibody (“anti-P-selectin antibody”) and b) at least one further therapeutic agent.

### BACKGROUND OF THE INVENTION

Myeloproliferative neoplasms (MPNs) are a unique and heterogeneous group of hemopathies characterized by proliferation and accumulation of mature myeloid cells, including myelofibrosis (MF), essential thrombocythemia (ET) and polycythemia vera (PV). Importantly, MF is the most severe form of Philadelphia chromosome-negative (i.e. BCR-ABL1-negative) myeloproliferative neoplasms, with a prevalence estimated to be 2.2 per 100,000 population. Myelofibrosis (MF) can present as a de novo disorder (PMF) or evolve from previous PV or ET (PPV-MF or PET-MF). The range of reported frequencies for post-PV MF are 4.9-6% at 10 years and 6-14% at 15 years, respectively, and 0.8-4.9% for post-ET MF at 10 years and 4-11% at 15 years, respectively (S Cerquozzi and A Tefferi, Blood Cancer Journal (2015) 5, e366).

Regardless of whether MF developed from PV, ET or as a primary disorder, it is characterized by a clonal stem cell proliferation associated with production of elevated levels of several inflammatory and proangiogenic cytokines resulting in a bone marrow stromal reaction that includes varying degrees of reticulin and/or collagen fibrosis, osteosclerosis and angiogenesis, some degree of megakaryocyte atypia and a peripheral blood smear showing a leukoerythroblastic pattern with varying degrees of circulating progenitor cells. The abnormal bone marrow milieu results in release of hematopoietic stem cells into the blood, extramedullary hematopoiesis, and organomegaly at these sites. Clinically, MF is characterized by progressive anemia, leukopenia or leukocytosis, thrombocytopenia or thrombocythemia and multi-organ extramedullary hematopoiesis, which most prominently involves the spleen leading to massive splenomegaly, severe constitutional symptoms, a hypermetabolic state, cachexia, and premature death.

A considerable number of cytokine and growth factor receptors utilize non-receptor tyrosine kinases, the Janus kinases (JAKs), to transmit extracellular ligand binding into an intracellular response. For example, erythropoietin, thrombopoietin and granulocyte monocyte colony stimulating factor are all known to signal through receptors that utilize JAK2. JAKs activate a number of downstream pathways implicated in proliferation and survival, including the STATs (signal transducers and activators of transcription), a family of important latent transcription factors.

Myelofibrosis is now known to be a clonal stem cell disease characterized by molecular (JAK2V617F, MPLW515L/K) and cytogenetic (13q-,20q-) markers (Pikman Y, Lee B H, Mercher T, et al. PLOS Med. 2006; 3(7):e270; Scott L M, Tong W, Levine R L, et al. N Engl J Med. 2007; 356:459-468). The JAK2V617F mutation has been identified in over 95% of patients with PV and approximately 50% of patients with ET and PMF. Furthermore, in a preclinical setting, animal studies have demonstrated that this mutation can lead to an MF-like syndrome. The JAK2V617F mutation alters the JAK2 tyrosine kinase making it constitutively active. As a result, polycythemia, thrombocythemia and leukocytosis can develop independently from growth factor regulation. Even in patients lacking a confirmed JAK2 mutation, the detection of STAT activation suggests dysregulated JAK activity. In fact, regardless of the mutational status of JAK2, the malignant cells appear to retain their responsiveness to JAK activating cytokines and/or growth factors; hence, they may benefit from JAK inhibition. Although several JAKs inhibitors, including ruxolitinib (brand name Jakavi) have been approved for the treatment of MF, they have only demonstrated effect in treatment of symptoms. Progression of the disease is not halted and eventually patients may die prematurely.

Therefore, there is a high unmet medical need to finding new and efficacious treatment options for advancing the treatment of myelofibrosis.

### SUMMARY OF THE INVENTION

It is an object of the present invention to provide for a medicament for the treatment of myelofibrosis. The present invention is based on the inventors' surprising finding that an anti-P-selectin antibody, or binding fragment thereof, suitably crizanlizumab or a binding fragment thereof, is useful in the treatment of myelofibrosis in a subject.

The present invention is also based on finding that an anti-P-selectin antibody, or binding fragment thereof, suitably crizanlizumab or a binding fragment thereof, in combination with at least one further therapeutic agent is useful in the treatment of myelofibrosis in a subject.

### DETAILED DESCRIPTION OF THE INVENTION

The term “anti-P-selectin antibody” as used herein refers to an antibody that is capable of binding to P-selectin specifically, i.e. it binds to P-selectin with an affinity higher than an antibody that is well known not to bind P-selectin specifically. The term “binding fragment” as used herein refers to a portion of an antibody that is capable of binding to P-selectin specifically. The affinity can be suitably determined by, for example, surface plasmon resonance (BIAcore™) assay. Ideally, the Kd of a P-selectin antibody or a fragment thereof is ≤1000 nM, or ≤500 nM, or ≤100 nM, or ≤50 nM, or more preferably by a Kd≤25 nM, and still more preferably by a Kd≤10 nM, and even more preferably by a Kd≤5 nM, or ≤1 nM, or ≤0.1 nM.

In one embodiment, the binding fragment may comprise an antigen binding and/or variable region. Merely by way of example, a suitable binding fragment may be selected from the group consisting of Fab, Fab′, F(ab′)2, Fv and scFv.

Suitably, the binding of the antibody (or binding fragment thereof) to P-selectin inhibits the binding of P-selectin to PSGL-1 and thereby reduces the formation of P-selectin/PSGL-1 complexes. Suitably, the anti-P-selectin antibody or binding fragment thereof may reduce the formation of P-selectin/PSGL-1 complexes by at least 30%, at least 40%, at least 50%, at least 60%, at least 70%, at least 80%, at least 90%, at least 95%, at least 96%, at least 97%, at least 98%, at least 99%, or more as compared to a suitable control (for example a sample without the presence of an anti-P-selectin antibody or binding fragment thereof).

Additionally or alternatively, an anti-P-selectin antibody or binding fragment thereof may dissociate preformed P-selectin/PSGL-1 complexes. In a suitable embodiment the anti-P-selectin antibody or binding fragment thereof may dissociate at least 30%, at least 40%, at least 50%, at least 60%, at least 70%, at least 80%, at least 90%, at least 95%, at least 96%, at least 97%, at least 98%, at least 99%, or more of preformed P-selectin/PSGL-1 complexes. As before, this property may be compared to a suitable control (for example a sample without the presence of an anti-P-selectin antibody or binding fragment thereof).

In one embodiment, the anti-P-selectin antibody or binding fragment thereof may bind P-selectin at any suitable epitope. Suitably, the anti-P-selectin antibody or binding fragment thereof may bind an epitope which is found in the P-selectin lectin-like domain.

In one embodiment, the anti-P-selectin antibody or binding fragment thereof binds P-selectin at amino acid positions 1 to 35 of SEQ ID NO: 1. Suitably the anti-P-selectin antibody or binding fragment thereof binds P-selectin at amino acid positions 4 to 23 of SEQ ID NO: 1. More suitably, the anti-P-selectin antibody or binding fragment thereof binds P-selectin at amino acid positions 4, 14, 17, 21, and 22 of SEQ ID NO: 1.

In one embodiment, the anti-P-selectin antibody or binding fragment thereof comprises a light chain variable region having a CDR sequence selected from the group consisting of KASQSVDYDGHSYMN (SEQ ID NO: 2), AASNLES (SEQ ID NO: 3) and QQSDENPLT (SEQ ID NO: 4).

In a suitable embodiment, the anti-P-selectin antibody or binding fragment thereof may comprise a light chain variable CDR with an amino acid sequence that varies from a sequence selected from the group consisting of KASQSVDYDGHSYMN (SEQ ID NO: 2), AASNLES (SEQ ID NO: 3) and QQSDENPLT (SEQ ID NO: 4) by no more than four amino acid residues, by no more than three amino acid residues, by no more than two amino acid residues, or by no more than one amino acid residue.

In one embodiment the anti-P-selectin antibody or binding fragment thereof comprises a light chain variable region comprising SEQ ID NO: 5.

In a suitable embodiment, the anti-P-selectin antibody or binding fragment thereof comprises a light chain variable region which comprises or consists of a polypeptide which is at least 90%, at least 95%, at least 96%, at least 97%, at least 98%, or at least 99% identical to SEQ ID NO: 5.

In one embodiment, the anti-P-selectin antibody or binding fragment thereof comprises a heavy chain variable region having a CDR sequence selected from the group consisting of SYDIN (SEQ ID NO: 6), WIYPGDGSIKYNEKFKG (SEQ ID NO: 7) and RGEYGNYEGAMDY (SEQ ID NO: 8).

In a suitable embodiment, the anti-P-selectin antibody or binding fragment thereof may comprise a heavy chain variable CDR with an amino acid sequence that varies from a sequence selected from the group consisting of SYDIN (SEQ ID NO: 6), WIYPGDGSIKYNEKFKG (SEQ ID NO: 7) and RGEYGNYEGAMDY (SEQ ID NO: 8) by no more than four amino acid residues, by no more than three amino acid residues, by no more than two amino acid residues, or by no more than one amino acid residue.

In one embodiment the anti-P-selectin antibody or binding fragment thereof comprises a heavy chain variable region comprising SEQ ID NO: 9.

In a suitable embodiment the anti-P-selectin antibody or binding fragment thereof comprises a heavy chain variable region which comprises or consists of a polypeptide which is at least 90%, at least 95%, at least 96%, at least 97%, at least 98%, or at least 99% identical to SEQ ID NO: 9.

In one embodiment the anti-P-selectin antibody or binding fragment thereof comprises a heavy chain variable region comprising three CDRs consisting essentially of or consisting of SEQ ID NO: 6, SEQ ID NO: 7, and SEQ ID NO: 8, respectively and a light chain variable region comprising three CDRs consisting essentially of or consisting of SEQ ID NO: 2, SEQ ID NO: 3, and SEQ ID NO: 4, respectively.

In one embodiment the anti-P-selectin antibody or binding fragment thereof comprises a light chain variable region comprising, consisting essentially of or consisting of the sequence SEQ ID NO: 5 and a heavy chain variable region comprising, consisting essentially of or consisting of the sequence SEQ ID NO: 9.

In one embodiment the anti-P-selectin antibody comprises a light chain which is at least 90%, at least 95%, at least 96%, at least 97%, at least 98%, at least 99% identical to SEQ ID NO: 10. Suitably, the anti-P-selectin antibody comprises a light chain according to SEQ ID NO: 10.

In one embodiment the anti-P-selectin antibody comprises a heavy chain which is at least 90%, at least 95%, at least 96%, at least 97%, at least 98%, at least 99% identical to SEQ ID NO: 11. Suitably, the anti-P-selectin antibody comprises a heavy chain according to SEQ ID NO: 11.

In a suitable embodiment the anti-P-selectin antibody comprises a light chain which is at least 90%, at least 95%, at least 96%, at least 97%, at least 98%, at least 99% identical to SEQ ID NO: 10, and a heavy chain which is at least 90%, at least 95%, at least 96%, at least 97%, at least 98%, at least 99% identical to SEQ ID NO: 11. Suitably the anti-P-selectin antibody comprises a light chain according to SEQ ID NO: 10, and a heavy chain according to SEQ ID NO: 11.

In one embodiment, the anti-P-selectin antibody or a binding fragment thereof is crizanlizumab or a binding fragment thereof.

In one embodiment, the anti-P-selectin antibody or binding fragment thereof may have a strong affinity to P-selectin. Suitably, the affinity of the antibody or binding fragment thereof to P-selectin, may be higher than the affinity of P-selectin to PSGL-1.

As used herein, the term “crizanlizumab” (formerly SelG1, registered with number 10316 in the International Nonproprietary Name (INN) database) refers to the anti-P-selectin antibody as described in WO2008/069999 and WO2012/088265, which are incorporated herein by reference. Crizanlizumab is a humanized monoclonal antibody targeted towards P-selectin and blocks its interaction with P-selectin glycoprotein ligand 1 (PSGL-1). In addition to blocking the interaction between P-selecting and PSGL-1, crizanlizumab also dissociates P-selectin/PSLG-1 complexes that have already formed.

Other suitable anti-P-selectin antibodies are disclosed in WO2005/100402, WO1993/021956 and WO1994/025067, which are hereby incorporated by reference in their entirety. In one embodiment, the suitable anti-P-selectin antibody or a fragment thereof is inclacumab or a binding fragment thereof.

As used herein, “ruxolitinib” is the JAK1/JAK2 inhibitor (R)-3-(4-(7H-pyrrolo[2,3-d]pyrimidin-4-yl)-1H-pyrazol-1-yl)-3-cyclopentylpropanenitrile, also named 3(R)-Cyclopentyl-3-[4-(7H-pyrrolo[2,3-d]pyrimidin-4-yl)-1H-pyrazol-1-yl]propanenitrile, of formula:

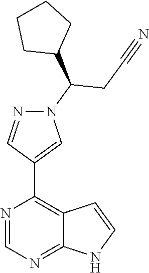 [SMILES: N#CC[C@H](C1CCCC1)n1cc(-c2ncnc3[nH]ccc23)cn1]

which can be prepared, for example, as described in WO2007/070514, which is incorporated herein by reference. As used herein, “ruxolitinib” refers to the free form, and any reference to “a pharmaceutically acceptable salt thereof” refers to “a pharmaceutically acceptable acid addition salt thereof”, in particular ruxolitinib phosphate, which can be prepared, for example, as described in WO2008/157208, which is incorporated herein by reference. Ruxolitinib is approved for the treatment of intermediate to high-risk myelofibrosis under the tradename Jakafi®/Jakavi®.

Ruxolitinib, or pharmaceutically acceptable salt thereof, in particular ruxolitinib phosphate, can be in a unit dosage form (e.g. tablet), which is administered orally.

In one embodiment, “ruxolitinib” is also intended to represent isotopically labeled forms. Isotopically labeled compounds have structures depicted by the formula above except that one or more atoms are replaced by an atom having a selected atomic mass or mass number. Isotopes that can be incorporated into ruxolitinib, for example, isotopes of hydrogen, namely the compound of formula:

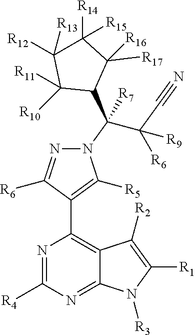

wherein each R₁, R₂, R₃, R₄, R₅, R₆, R₇, R₈, R₉, R₁₀, R₁₁, R₁₂, R₁₃, R₁₄, R₁₅, R₁₆ and R₁₇ is independently selected from H or deuterium; provided that there is at least one deuterium present in the compound. In other embodiments there are multiple deuterium atoms present in the compound. Suitable compounds are disclosed in U.S. Pat. No. 9,249,149 B2, which is hereby incorporated in its entirety.

In one preferred embodiment, a deuterated ruxolitinib is selected from the group consisting of

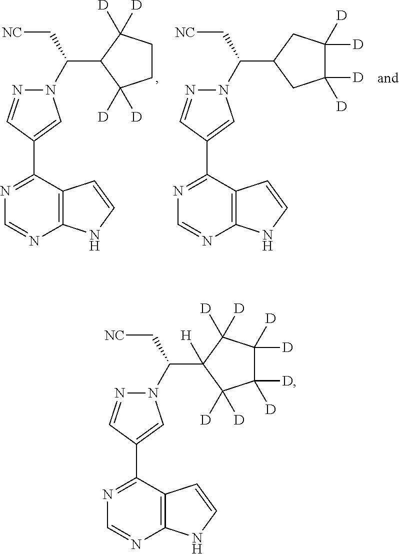

or a pharmaceutically acceptable salt of any of the foregoing.

In a preferred embodiment, a deuterated ruxolitinib is

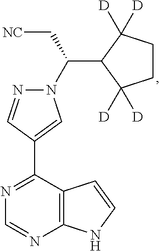 [SMILES: [2H]C1([2H])CCC([2H])([2H])C1[C@@H](CC#N)n1cc(-c2ncnc3[nH]ccc23)cn1]

or a pharmaceutically acceptable salt thereof.

As used herein, “itacitinib” refers to the JAK1/JAK2 inhibitor 2-(3-(4-(7H-pyrrolo(2,3-d)pyrimidin-4-yl)-1H-pyrazol-1-yl)-1-(1-(3-fluoro-2-(trifluoromethyl)isonicotinoyl)piperidin-4-yl)azetidin-3-yl)acetonitrile, also named 2-[1-[1-[3-fluoro-2-(trifluoromethyl)pyridine-4-carbonyl]piperidin-4-yl]-3-[4-(7H-pyrrolo[2,3-d]pyrimidin-4-yl)pyrazol-1-yl]azetidin-3-yl]acetonitrile of formula

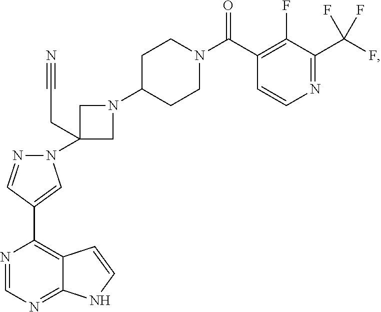 [SMILES: N#CCC1(n2cc(-c3ncnc4[nH]ccc34)cn2)CN(C2CCN(C(=O)c3ccnc(C(F)(F)F)c3F)CC2)C1]

which can be prepared, for example, as described in WO2011/112662, which is incorporated herein by reference. As used herein, “itacitinib” refers to the free form, and any reference to “a pharmaceutically acceptable salt thereof” refers to “a pharmaceutically acceptable acid addition salt thereof”, in particular itacitinib adipate.

### Treatment of Myelofibrosis

Increased megakaryocyte (MK) proliferation in bone marrow is commonly observed in Philadelphia-chromosome negative MPN. In MF patients megakaryocytes are observed to have increased P-selectin on their intracytoplasmic vacuoles and demarcation membrane system (DMS), leading to increased emperipolesis (the passage of a cell into the cytoplasm of another cell) of neutrophils. These neutrophils release their enzymes in the megakaryocytes leading to the release of cytokines such as transforming growth factor beta (TGF-β), platelet derived growth factor (PDGF) and fibroblast growth factor (FGF) from their alpha granules (Schmitt A, Jouault H, Guichard J, et al. Blood 2000; 96:1342-7). Once released, the growth factors stimulate the deposition of reticulin and collagen fibers by fibroblasts, and increased production of osteoprotegerin by stromal and endothelial cells leading to unbalanced osteoblast proliferation, resulting in osteosclerosis and neoangiogenesis (Cervantes F, Martinez-Trillos A. Expert Opin Pharmacother 2013; 14:873-84; Chagraoui H, Tulliez M, Smayra T, et al. Blood 2003; 101:2983-9). Moreover, studies on Gata1^{low} mice, a mouse model of myelofibrosis, have shown that genetic deletion of the P-selectin gene (P-sel) reduced thrombotic events and progression from the pre-fibrotic stage into the fibrotic stage (Spangrude et al, Stem Cells, 2016, 34: 67-82).

Thus, in one aspect the present invention provides an anti-P-selectin antibody or binding fragment thereof, suitably crizanlizumab or a binding fragment thereof, alone or in combination with a JAK inhibitor, suitably ruxolitinib or a pharmaceutical acceptable salt thereof, for use in the treatment of Philadelphia-chromosome negative myeloproliferative neoplasms.

In one further aspect the present invention provides an anti-P-selectin antibody or binding fragment thereof, suitably crizanlizumab or a binding fragment thereof, for use in the treatment of myelofibrosis (MF) in a patient. Alternatively, in one aspect the present invention provides an anti-P-selectin antibody or binding fragment thereof, suitably crizanlizumab or a binding fragment thereof, for use in the manufacture of a medicament for the treatment of myelofibrosis (MF) in a patient. Alternatively, in one aspect the present invention provides a method of treating myelofibrosis (MF) in a patient comprising the step of administering therapeutically effective amount of an anti-P-selectin antibody or binding fragment thereof, suitably crizanlizumab or a binding fragment thereof, to said patient.

Myelofibrosis comprises primary myelofibrosis (PMF), post-essential thrombocythemia myelofibrosis (PET-MF) and post-polycythemia vera myelofibrosis (PPV-MF). Suitably, myelofibrosis is PMF.

The term “primary myelofibrosis” (PMF), as used herein, is defined with reference to “The 2016 revision to the World Health Organization (WHO) classification of myeloid neoplasms and acute leukemia”, as published in Blood, 2016, 127:2391-2405. Primary myelofibrosis encompasses prefibrotic/early primary myelofibrosis (prePMF) and overt primary myelofibrosis (overt PMF). Diagnosis of prePMF requires meeting the following 3 major criteria, and at least 1 minor criterion according to the 2016 WHO classification for prePMF in table 1:

TABLE 1
Criteria for diagnosis of prePMF

Major criteria (prePMF)
1. Megakaryocytic proliferation and atypia, without reticulin fibrosis >grade 1, accompanied by
increased age-adjusted BM cellularity, granulocytic proliferation, and often decreased erythropoiesis
2. Not meeting the WHO criteria for BCR-ABL1⁺CML, PV, ET, myelodysplastic syndromes, or other
myeloid neoplasms
3. Presence of JAK2, CALR, or MPL mutation or in the absence of these mutations, presence of
another clonal marker (e.g., ASXL1, EZH2, TET2, IDH1/IDH2, SRSF2, SF3B1) are of help in
determining the clonal nature of the disease or absence of minor reactive bone marrow (BM) reticulin
fibrosis (Minor (grade 1) reticulin fibrosis secondary to infection, autoimmune disorder or other chronic
inflammatory conditions, hairy cell leukemia or other lymphoid neoplasm, metastatic malignancy, or
toxic (chronic) myelopathies)
Minor criteria (prePMF)
Presence of at least 1 of the following, confirmed in 2 consecutive determinations:
a. Anemia not attributed to a comorbid condition
b. Leukocytosis ≥11*10⁹/L
c. Palpable splenomegaly
d. LDH increased to above upper normal limit of institutional reference range

Diagnosis of overt PMF requires meeting the following 3 major criteria, and at least 1 minor criterion according to the 2016 WHO classification for overt PMF in table 2:

TABLE 2
Criteria for diagnosis of overt PMF

Major criteria (overt PMF)
1. Presence of megakaryocytic proliferation and atypia, accompanied by either reticulin
and/or collagen fibrosis grades 2 or 3
2. Not meeting WHO criteria for ET, PV, BCR-ABL1⁺CML, myelodysplastic syndromes,
or other myeloid neoplasms
3. Presence of JAK2, CALR, or MPL mutation or in the absence of these mutations, presence of another
clonal marker (e.g., ASXL1, EZH2, TET2, IDH1/IDH2, SRSF2, SF3B1) or absence of reactive
myelofibrosis (BM fibrosis secondary to infection, autoimmune disorder, or other chronic inflammatory
conditions, hairy cell leukemia or other lymphoid neoplasm, metastatic malignancy, or toxic (chronic)
myelopathies)
Minor criteria (overt PMF)
Presence of at least 1 of the following, confirmed in 2 consecutive determinations:
a. Anemia not attributed to a comorbid condition
b. Leukocytosis ≥11*10⁹/L
c. Palpable splenomegaly
d. LDH increased to above upper normal limit of institutional reference range
e. Leukoerythroblastosis

The term “bone marrow fibrosis”, as used herein, refers to bone marrow fibrosis graded according to the 2005 European consensus grading system (Thiele et. al., Haematologica, 2005, 90(8), 1128-1132, in particular as defined in Table 3 and FIG. 1 of page 1130 therein), such as:
“fibrosis grade 0”: scattered linear reticulin with no intersections (cross-overs) corresponding to normal bone marrow;
“fibrosis grade 1”: loose network of reticulin with many intersections, especially in perivascular areas;
“fibrosis grade 2”: diffuse and dense increase in reticulin with extensive intersections, occasionally with only focal bundles of collagen and/or focal osteosclerosis;
“fibrosis grade 3”: diffuse and dense increase in reticulin with extensive intersections with coarse bundles of collagen, often associated with significant osteosclerosis;
wherein the grading (i.e. grading of fiber density and quality) is made on the basis of bone marrow biopsy specimen assessment.

The term “essential thrombocythemia” (ET), as used herein, is defined with reference to “The 2016 revision to the World Health Organization (WHO) classification of myeloid neoplasms and acute leukemia”, as published in Blood, 2016, 127:2391-2405. The term “post-essential thrombocythemia myelofibrosis” (PET-MF), as used herein, refers to MF secondary to ET (i.e. MF arising as a progression of ET), wherein ET is as defined herein above. According to the IWG-MRT criteria (Barosi G et al, Leukemia (2008) 22, 437-438), criteria for diagnosing post-essential thrombocythemia myelofibrosis are:

TABLE 3
Criteria for diagnosis of post-essential thrombocythemia myelofibrosis

Required criteria:
1. Documentation of a previous diagnosis of essential thrombocythemia as defined by the WHO criteria
2. Bone marrow fibrosis grade 2-3
Additional criteria (two are required):
1. Anemia and a ≥2 mg/ml decrease from baseline hemoglobin level
2. A leukoerythroblastic peripheral blood picture
3. Increasing splenomegaly defined as either an increase in palpable splenomegaly of ≥5cm (distance of
the tip of the spleen from the left costal margin) or the appearance of a newly palpable splenomegaly
4. Increased lactate dehydrogenase (LDH) (above reference level)
5. Development of ≥1 of three constitutional symptoms: >10% weight loss in 6 months, night sweats,
unexplained fever (>37.5° C.)

The term “polycythemia vera” (PV), as used herein, is defined with reference to “The 2016 revision to the World Health Organization (WHO) classification of myeloid neoplasms and acute leukemia”, as published in Blood, 2016, 127:2391-2405. The term “post-polycythemia myelofibrosis” (PPV-MF), as used herein, refers to MF secondary to PV (i.e. MF arising as a progression of PV). According to the IWG-MRT criteria (Barosi G et al, Leukemia (2008) 22, 437-438), criteria for diagnosing post-polycythemia myelofibrosis are:

TABLE 4
Criteria for diagnosis of post-polycythemia myelofibrosis

Required criteria:
1. Documentation of a previous diagnosis of polycythemia vera as defined by the WHO criteria
2. Bone marrow fibrosis grade 2-3 (on 0-3 scale)
Additional criteria (two are required):
1. Anemia or sustained loss of requirement of either phlebotomy (in the absence of cytoreductive
therapy) or cytoreductive treatment for erythrocytosis
2. A leukoerythroblastic peripheral blood picture
3. Increasing splenomegaly defined as either an increase in palpable splenomegaly of ≥5 cm (distance of
the tip of the spleen from the left costal margin) or the appearance of a newly palpable splenomegaly
4. Development of ≥1 of three constitutional symptoms: >10% weight loss in 6 months, night sweats,
unexplained fever (>37.5° C.)

As used herein, the following response criteria as defined by the International Working Group-Myeloproliferative Neoplasms Research and Treatment (IWG-MRT) and the European Leukemia Net (ELN) response criteria for MF (Tofferi et al, Blood 2013 122:1395-1398, which is incorporated by reference in its entirety) are used herein:

TABLE 5
International Working Group-Myeloproliferative Neoplasms Research and Treatment
(IWG-MRT) and the European Leukemia Net (ELN) response criteria for myelofibrosis

| Response | Required criteria (for all response categories, |
|---|---|
| categories | benefit must last for ≥12 weeks to qualify as a response) |
| Complete | Bone marrow:* Age-adjusted normocellularity; <5% blasts; ≤grade 1 MF^{†} and |
| response (CR) | Peripheral blood: Hemoglobin ≥100 g/L and <UNL; neutrophil |
| | count ≥1 × 10⁹/L and <UNL; |
| | Platelet count ≥100 × 10⁹/L and <UNL; <2% immature myeloid cells^{‡} and |
| | Clinical: Resolution of disease symptoms; spleen and liver not palpable; no |
| | evidence of EMH |
| Partial | Peripheral blood: Hemoglobin ≥100 g/L and <UNL; neutrophil count ≥1 × 10⁹/L and <UNL; |
| response (PR) | platelet count ≥100 × 10⁹/L and <UNL; <2% immature myeloid cells^{‡} and |
| | Clinical: Resolution of disease symptoms; spleen and liver not palpable; no |
| | evidence of EMH or |
| | Bone marrow:* Age-adjusted normocellularity; <5% blasts; ≤grade 1 MF^{†}, and |
| | peripheral blood: Hemoglobin ≥85 but <100 g/L and <UNL; neutrophil |
| | count ≥1 × 10⁹/L and <UNL; platelet count ≥50, but <100 × 10⁹/L and <UNL; <2% |
| | immature myeloid cells^{‡} and |
| | Clinical: Resolution of disease symptoms; spleen and liver not palpable; no |
| | evidence of EMH |
| Clinical | The achievement of anemia, spleen or symptoms response without progressive |
| improvement | disease or increase in severity of anemia, thrombocytopenia, or neutropenia^{§} |
| (CI) | |
| Anemia | Transfusion-independent patients: a ≥20 g/L increase in hemoglobin level^{\|\|} |
| response | Transfusion-dependent patients: becoming transfusion-independent^{¶} |
| Spleen | A baseline splenomegaly that is palpable at 5-10 cm, below the LCM, becomes not |
| response^{#} | palpable** or |
| | A baseline splenomegaly that is palpable at >10 cm, below the LCM, |
| | decreases by ≥50%** |
| | A baseline splenomegaly that is palpable at <5 cm, below the LCM, is not eligible for |
| | spleen response |
| | A spleen response requires confirmation by MRI or computed tomography |
| | showing ≥35% spleen volume reduction |
| Symptoms | A ≥50% reduction in the MPN-SAF TSS^{††} |
| response | |

EMH, extramedullary hematopoiesis (no evidence of EMH implies the absence of pathology- or imaging study-proven nonhepatosplenic EMH); LCM, left costal margin; UNL, upper normal limit.
*Baseline and posttreatment bone marrow slides are to be interpreted at one sitting by a central review process.
^{†}Grading of MF is according to the European classification: Thiele et al. European consensus on grading bone marrow fibrosis and assessment of cellularity. Haematologica. 2005; 90: 1128.
^{‡}Immature myeloid cells constitute blasts + promyelocytes + myelocytes + metamyelocytes + nucleated red blood cells. In splenectomized patients, <5% immature myeloid cells is allowed.
^{§}Increase in severity of anemia constitutes the occurrence of new transfusion dependency or a ≥20 g/L decrease in hemoglobin level from pretreatment baseline that lasts for at least 12 weeks. Increase in severity of thrombocytopenia or neutropenia is defined as a 2-grade decline, from pretreatment baseline, in platelet count or absolute neutrophil count, according to the Common Terminology Criteria for Adverse Events (CTCAE) version 4.0. In addition, assignment to CI requires a minimum platelet count of ≥25 000 × 10(9)/L and absolute neutrophil count of ≥0.5 × 10(9)/L.
^{||}Applicable only to patients with baseline hemoglobin of <100 g/L. In patients not meeting the strict criteria for transfusion dependency at the time of treatment initiation, but have received transfusions within the previous month, the pre-transfusion hemoglobin level should be used as the baseline.
^{¶}Transfusion dependency is defined as transfusions of at least 6 units of packed red blood cells (PRBC), in the 12 weeks prior to start of treatment initiation, for a hemoglobin level of <85 g/L, in the absence of bleeding or treatment-induced anemia. In addition, the most recent transfusion episode must have occurred in the 28 days prior to start of treatment initiation. Response in transfusion-dependent patients requires absence of any PRBC transfusions during any consecutive “rolling” 12-week interval during the treatment phase, capped by a hemoglobin level of ≥85 g/L.
^{#}In splenectomized patients, palpable hepatomegaly is substituted with the same measurement strategy.
**Spleen or liver responses must be confirmed by imaging studies where a ≥35% reduction in spleen volume, as assessed by MRI or CT, is required. Furthermore, a ≥35% volume reduction in the spleen or liver, by MRI or CT, constitutes a response regardless of what is reported with physical examination.
^{††}Symptoms are evaluated by the MPN-SAF TSS. The MPN-SAF TSS is assessed by the patients themselves and this includes fatigue, concentration, early satiety, inactivity, night sweats, itching, bone pain, abdominal discomfort, weight loss, and fevers. Scoring is from 0 (absent/as good as it can be) to 10 (worst imaginable/as bad as it can be) for each item. The MPN-SAF TSS is the summation of all the individual scores (0-100 scale). Symptoms response requires ≥50% reduction in the MPN-SAF TSS.

In one embodiment the present invention provides crizanlizumab or a binding fragment thereof, alone or in combination with a JAKs inhibitor, suitably ruxolitinib or a pharmaceutically acceptable salt thereof, for use in the treatment of myelofibrosis, especially primary MF, wherein the patient achieves complete response to the treatment according to the criteria in Table 5.

In one embodiment the present invention provides crizanlizumab or a binding fragment thereof, alone or in combination with a JAKs inhibitor, suitably ruxolitinib or a pharmaceutically acceptable salt thereof, for use in the treatment of myelofibrosis, especially primary MF, wherein the patient achieves partial response to the treatment according to the criteria in Table 5.

Among patients, myelofibrosis frequently causes shortened survival due to disease transformation to acute leukemia, progression without acute transformation, cardiovascular complications or thrombosis, infection or portal hypertension. It is one of the aims of the present invention to improve the median survival of myelofibrosis patients.

As used herein, the term “median survival time” refers to the time of diagnosis or from the time of initiation of treatment according to the present invention that half of the patients in a group of patients diagnosed with the disease are still alive compared to patients receiving best available treatment or compared to patients receiving placebo and wherein patients belong to the same risk group of myelofibrosis, for example as described by Gangat et al (J Clin Oncol. 2011 Feb. 1; 29(4):392-397), which is hereby incorporated by reference in its entirety.

Accordingly, in one embodiment the present invention provides an anti-P-selectin antibody or binding fragment thereof, suitably crizanlizumab or a binding fragment thereof, alone or in combination with a JAKs inhibitor, suitably ruxolitinib or a pharmaceutically acceptable salt thereof, for use in the treatment of myelofibrosis, especially primary MF, wherein median survival time is increased by at least 3 months in the group of high risk MF patients or by at least six months, preferably by at least 12 months in the group of medium risk MF patients.

As used herein, the term “subject” refers to a human being.

The term “treat”, “treating”, “treatment” or “therapy”, as used herein, means obtaining beneficial or desired results, for example, clinical results. Beneficial or desired results can include, but are not limited to, alleviation of one or more symptoms, as defined herein. One aspect of the treatment is, for example, that said treatment should have a minimal adverse effect on the patient, e.g. the agent used should have a high level of safety, for example without producing the side effects of a previously known therapy. The term “alleviation”, for example in reference to a symptom of a condition, as used herein, refers to reducing at least one of the frequency and amplitude of a symptom of a condition in a patient.

As used herein, the term “newly diagnosed” refers to diagnosis of the disorder, e.g. myelofibrosis and said patient has not received any treatment. In one embodiment the present invention provides an anti-P-selectin antibody or binding fragment thereof, suitably crizanlizumab or a binding fragment thereof, alone or in combination with a JAK inhibitor, suitably ruxolitinib or a pharmaceutically acceptable salt thereof, for use in the treatment of a newly diagnosed myelofibrosis patient

The term “triple-negative myelofibrosis patient”, as used herein, refers to a patient who lacks JAK2, CALR and MPL mutations. In one embodiment the present invention provides an anti-P-selectin antibody or binding fragment thereof, suitably crizanlizumab or a binding fragment thereof, alone or in combination with a JAK inhibitor, suitably ruxolitinib or a pharmaceutically acceptable salt thereof, for use in the treatment of triple-negative myelofibrosis patient.

The term “best available therapy”, as used herein, refers to any commercially available agent approved prior to March 2018 for the treatment of PMF, PET-MF or PPV-MF, as monotherapy, or in combination. Exemplary agents include, but are not limited to ruxolitinib or a pharmaceutically acceptable salt thereof, antineoplastic agents (e.g., hydroxyurea, anagrelide), glucocorticoids (e.g., prednisone/prednisolone, methylprednisolone), antianemia preparations (e.g., epoetin-alpha), immunomodulatory agents (e.g., thalidomide, lenalidomide), purine analogs (e.g., mercaptopurine, thioguanine), antigonadotropins (e.g., danazol), interferons (e.g., PEG-interferon-alpha 2a, interferon-alpha), nitrogen mustard analogs (e.g. melphalan), pyrimidine analogs (e.g., cytarabine).

The term “splenomegaly”, as used herein, refers to a palpably enlarged spleen (e.g. a spleen is palpable at ≥5 cm below the left coastal margin) or to an enlarged spleen as detected by an imaging test (e.g. a computed tomography (CT) scan, MRI, X-rays or ultrasound), wherein the term “enlarged spleen” refers to a spleen greater in size than normal (e.g., median normal spleen volume of 200 cm³).

The term “treatment of splenomegaly”, as used herein, refers to “improvement of splenomegaly”, which means a decrease in splenomegaly, for example a reduction in spleen volume, as defined by the International Working Group-Myeloproliferative Neoplasms Research and Treatment (IWG-MRT) and the European Leukemia Net (ELN) response criteria for MF in Table 5. In one embodiment, the invention may provide the use of an anti-P-selectin antibody or binding fragment thereof, suitably crizanlizumab or a binding fragment thereof, alone or in combination with ruxolitinib or a pharmaceutically acceptable salt thereof for treatment of myelofibrosis, particularly for the treatment of splenomegaly associated with myelofibrosis, resulting in, for example, ≥20%, ≥25%, ≥30% or ≥35% reduction in spleen volume as measured by magnetic resonance imaging (MRI) or computed tomography (CT) from pre-treatment baseline to, for example, week 24 or week 48.

The term “hepatomegaly”, as used herein, refers to a palpably enlarged liver or to an enlarged liver as detected by an imaging test (e.g. a computed tomography (CT) scan), wherein the term “enlarged liver” refers to a liver greater in size than normal (e.g., median normal liver volume of approximately 1500 cm³).

The term “treatment of hepatomegaly”, as used herein, refers to “improvement of hepatomegaly”, which means a decrease in hepatomegaly, for example a reduction in hepatomegaly, as defined according to the International Working Group-Myeloproliferative Neoplasms Research and Treatment (IWG-MRT) and the European Leukemia Net (ELN) response criteria for MF in the preceding table. Accordingly, in one embodiment the present invention provides the use of an anti-P-selectin antibody or binding fragment thereof, suitably crizanlizumab or a binding fragment thereof, alone or in combination with ruxolitinib or a pharmaceutically acceptable salt thereof for treatment of myelofibrosis, particularly for the treatment of hepatomegaly associated with myelofibrosis, resulting in, for example, ≥20%, ≥25%, ≥30% or ≥35% reduction in liver volume as measured by magnetic resonance imaging (MRI) or computed tomography (CT) from pre-treatment baseline to, for example, week 24 or week 48.

The term “thrombocytopenia”, as used herein, refers to a platelet count, in blood specimen laboratory test, lower than normal. The term “severity of thrombocytopenia”, as used herein, refers, for example, to specific grade 1-4 of thrombocytopenia according to CTCAE (version 4.03).

The term “treatment of thrombocytopenia”, as used herein, refers to “stabilizing thrombocytopenia” or “improving thrombocytopenia”, in comparison to the pre-treatment situation or in comparison to best available therapy or to placebo control. The term “stabilizing thrombocytopenia” refers, for example, to prevent an increase in the severity of thrombocytopenia, namely the platelet count remains stable. The term “improving thrombocytopenia” refers to alleviation of the severity of thrombocytopenia, namely increasing blood platelet count. In one embodiment, the invention provides an anti-P-selectin antibody or binding fragment thereof, suitably crizanlizumab or a binding fragment thereof, alone or in combination with ruxolitinib or a pharmaceutically acceptable salt thereof, for use in the treatment of myelofibrosis, particularly for the treatment of thrombocytopenia associated with myelofibrosis, resulting in stabilizing thrombocytopenia or improving thrombocytopenia from pre-treatment baseline to, for example, week 24 or week 48 of treatment.

The term “neutropenia”, as used herein, refers to an absolute neutrophil count (ANC), in blood specimen laboratory test, lower than normal value. The term “severity of neutropenia”, as used herein, refers, for example, to specific grade 1-4 of neutropenia according to CTCAE (version 4.03).

The term “treatment of neutropenia”, as used herein, refers to “stabilizing neutropenia” or “improving neutropenia”, for example, in comparison to the pre-treatment situation or in comparison to best available therapy or to placebo control. The term “stabilizing neutropenia” refers, for example, to prevent an increase in the severity of neutropenia. The term “improving neutropenia” refers, for example, to a decrease in the severity of neutropenia. In one embodiment, the invention provides an anti-P-selectin antibody or binding fragment thereof, suitably crizanlizumab or a binding fragment thereof, alone or in combination with ruxolitinib or a pharmaceutically acceptable salt thereof, for use in the treatment of myelofibrosis, particularly for the treatment of neutropenia associated with myelofibrosis, resulting in stabilizing neutropenia or improving neutropenia from pre-treatment baseline to, for example, week 24 or week 48 of treatment.

The term “anemia”, as used herein, refers to hemoglobin level, in blood specimen laboratory test, of less than 13.5 gram/100 ml in men and hemoglobin level of less than 12.0 gram/100 ml in women. The term “severity of anemia”, as used herein, refers, for example, to specific grade 1-4 of anemia according to CTCAE (version 4.03)].

The term “treatment of anemia”, as used herein, refers to “stabilizing anemia” or “improving anemia”, for example, in comparison to the pre-treatment situation or in comparison to best available therapy or to placebo control. The term “stabilizing anemia” refers, for example, to prevent an increase in the severity of anemia (e.g. preventing that a “transfusion-independent” patient becomes a “transfusion-dependent” patient or preventing anemia grade 2 becomes anemia grade 3). The term “improving anemia” refers to a decrease in the severity of anemia or an improvement in hemoglobin level. In one embodiment, the invention may provide the use of an anti-P-selectin antibody or binding fragment thereof, suitably crizanlizumab or a binding fragment thereof, alone or in combination with ruxolitinib or a pharmaceutically acceptable salt thereof, for treatment of myelofibrosis, particularly for the treatment of anemia associated with myelofibrosis, resulting in stabilizing anemia or improving anemia from pre-treatment baseline to, for example, week 24 or week 48 of treatment.

The term “treatment of bone marrow fibrosis associated with MF”, as used herein, means “stabilizing bone marrow fibrosis” or “improving bone marrow fibrosis”, for example, in comparison to the pre-treatment situation or in comparison to best available therapy or to placebo control. The term “stabilizing bone marrow fibrosis” refers, for example, to prevent increase in severity of bone marrow fibrosis. The term “improving bone marrow fibrosis” refers to a decrease in severity of bone marrow fibrosis, for example, from pre-treatment baseline, according to the 2005 European consensus grading system. In one embodiment, the invention may provide the use of an anti-P-selectin antibody or binding fragment thereof, suitably crizanlizumab or a binding fragment thereof, alone or in combination with ruxolitinib or a pharmaceutically acceptable salt thereof, for treatment of myelofibrosis, particularly for the treatment of bone marrow fibrosis associated with MF, resulting in stabilizing bone marrow fibrosis or improving bone marrow fibrosis from pre-treatment baseline to, for example, week 24 or week 48 of treatment.

The term “constitutional symptoms associated with myelofibrosis”, as used herein, refers to common debilitating chronic myelofibrosis symptoms, such as fever, pruritus (i.e. itching), abdominal pain/discomfort, weight loss, fatigue, inactivity, early satiety, night sweats or bone pain; for example, as described by Mughal et al (Int J Gen Med. 2014 Jan. 29; 7:89-101).

The term “treatment of constitutional symptoms associated with myelofibrosis”, as used herein, refers to “improvement of constitutional symptoms associated with myelofibrosis”, for example, in comparison to the pre-treatment situation or in comparison to best available therapy or to placebo control, for example, a reduction in total symptom score as measured by the modified myelofibrosis symptom assessment form version 2.0 diary (modified MFSAF v2.0) (Cancer 2011; 117:4869-77; N Engl J Med 2012; 366:799-807, the entire contents of which are incorporated herein by reference). In one embodiment, the invention may provide the use of an anti-P-selectin antibody or binding fragment thereof, suitably crizanlizumab or a binding fragment thereof, alone or in combination with ruxolitinib or a pharmaceutically acceptable salt thereof, for treatment of myelofibrosis, particularly for the treatment of constitutional symptoms associated with myelofibrosis, resulting in improvement of constitutional symptoms associated with myelofibrosis from pre-treatment baseline to, for example, week 24 or week 48 of treatment.

In another embodiment of any use of the invention, one or more of the constitutional symptoms associated with MF are alleviated (e.g. by eliminating or by reducing intensity, duration or frequency). In one embodiment, the reduction of constitutional symptoms is at least ≥20%, at least ≥30%, at least ≥40% or at least ≥50% as assessed by the modified MFSAF v2.0 from pre-treatment baseline to, for example, week 24 or week 48.

In one embodiment of any use of the invention, the anti-P-selectin antibody, or binding fragment thereof, suitably crizanlizumab or a binding fragment thereof, is administered subsequently or prior to splenectomy or radiotherapy, such as splenic irradiation.

### Combination Therapy

In one aspect the present invention provides an anti-P-selectin antibody or binding fragment thereof, suitably crizanlizumab or a binding fragment thereof, for use in the treatment of MF, wherein said P-selectin antibody, or binding fragment thereof, is administered in combination with at least one further active agent.

In one embodiment the at least one agent is an inhibitor of a non-receptor tyrosine kinases, the Janus kinases (JAKs). A considerable number of cytokine and growth factor receptors utilize non-receptor tyrosine kinases, the Janus kinases (JAKs), to transmit extracellular ligand binding into an intracellular response. For example, erythropoietin, thrombopoietin and granulocyte monocyte colony stimulating factor are all known to signal through receptors that utilize JAK2. JAKs activate a number of downstream pathways implicated in proliferation and survival, including the STATs (signal transducers and activators of transcription), a family of important latent transcription factors.

Accordingly, the present invention relates to the combination use of an anti-P-selectin antibody or binding fragment thereof, suitably crizanlizumab or a binding fragment thereof, with at least one JAKs inhibitor, suitably ruxolitinib or a pharmaceutically acceptable salt thereof.

In one embodiment the at least one further active agent is a JAK1/JAK2 inhibitor, suitably ruxolitinib or a pharmaceutically acceptable salt thereof or momelotinib or a pharmaceutically acceptable salt thereof, more suitably ruxolitinib or a pharmaceutically acceptable salt, more suitably ruxolitinib phosphate.

Ruxolitinib represents a novel, potent, and selective inhibitor of JAK1 and JAK2. Ruxolitinib potently inhibits JAK1 and JAK2 [half maximal inhibitory concentration (IC50) 0.4 to 1.7 nM], yet it does not significantly inhibit (<30% inhibition) a broad panel of 26 kinases when tested at 200 nM (approximately 100× the average IC50 value for JAK enzyme inhibition) and does not inhibit JAK3 at clinically relevant concentrations.

In one embodiment the at least one further active agent is a JAK2/FLT3 inhibitor, suitably pacritinib or a pharmaceutically acceptable salt thereof or fedratinib or a pharmaceutically acceptable salt thereof.

In one embodiment the at least one further active agent is a JAK2V617F inhibitor, suitably gandotinib or a pharmaceutically acceptable salt thereof.

In one embodiment the at least one further active agent is a JAK2 inhibitor, suitably BMS-911543 or a pharmaceutically acceptable salt thereof.

In one embodiment the at least one further active agent is a JAK1 inhibitor, suitably itacitinib or a pharmaceutically acceptable salt thereof, in particular itacitinib adipate.

In one embodiment the at least one further active agent is a JAK2/Src inhibitor, suitably NS-018 or a pharmaceutically acceptable salt thereof.

In one aspect the present invention provides a pharmaceutical combination, separate, comprising, consisting essentially of or consisting of a) crizanlizumab or a binding fragment thereof and b) a JAK1/2 inhibitor, suitably ruxolitinib or a pharmaceutically acceptable salt thereof. Suitably the pharmaceutical combination is for use in the treatment of myelofibrosis.

In one aspect the present invention provides crizanlizumab or a binding fragment thereof, for use in the treatment of myelofibrosis, wherein crizanlizumab or a binding fragment thereof, is administered in combination with ruxolitinib or a pharmaceutically acceptable salt thereof, and wherein crizanlizumab or a binding fragment thereof, and ruxolitinib or a pharmaceutically acceptable salt thereof, are administered in jointly therapeutically effective amounts.

In one aspect the present invention provides ruxolitinib or a pharmaceutically acceptable salt thereof, for use in the treatment of myelofibrosis, wherein ruxolitinib or a pharmaceutically acceptable salt thereof, is administered in combination with crizanlizumab or a binding fragment thereof, and wherein ruxolitinib or a pharmaceutically acceptable salt thereof, and crizanlizumab or a binding fragment thereof, are administered in jointly therapeutically effective amounts.

In one embodiment the present invention provides an anti-P-selectin antibody or binding fragment thereof, suitably crizanlizumab or a binding fragment thereof, for use in the treatment of myelofibrosis, wherein said P-selectin antibody, or binding fragment thereof, is administered in combination with at least one further active agent, wherein said at least one further active agent is selected from the group consisting of an HSP90 inhibitor (e.g. PU-H71, luminespib, ganatespib); an HDAC inhibitor (e.g. panobinostat, givinostat, pracinostat, vorinostat); a DNA methyltransferase inhibitor (e.g. 5-azacytidine, decitabine); an mTOR inhibitor (e.g. rapamycin, everolimus); an AKT inhibitor (e.g. MK-2206); a PI3K inhibitor (e.g. buparlisib, dactolisib); a Hedgehog inhibitor (e.g. glasdegib, saridegib, erismodegib); an SMO inhibitor (e.g. sonidegib, vismodegib); an anti-fibrotic agent, such as simtuzumab, serum amyloid P or a monoclonal antibody (e.g. fresolimumab, simtuzumab); an Aurora-A kinase inhibitor (e.g. dimetylfasudil, alisertib); a TNF-alpha modulator (e.g. danazol); an immunomodulatory agent (e.g. lenalidomide, pomalidomide, thalidomide); a glucocorticoid (e.g. prednisone); a telomerase inhibitor (e.g. imetelstat); an anti-anemics agent (e.g. an erythropoiesis stimulating agent such as sotatercept); a CYP3A4 inhibitor (e.g. ketoconazole, clarithromycin, itraconazole, nefazodone, telithromycin); and a dual CYP2C9-CYP3A4 inhibitor (e.g. fluconazole); or, in each case, a pharmaceutically acceptable salt thereof.

In one embodiment the present invention provides an anti-P-selectin antibody or binding fragment thereof, suitably crizanlizumab or a binding fragment thereof, for use in the treatment of myelofibrosis, wherein said P-selectin antibody, or binding fragment thereof, is administered in combination with at least one further active agent, wherein said at least one further active agent is a JAKs inhibitor, suitably ruxolitinib or a pharmaceutically acceptable salt thereof, and at least one further active agent selected from the group consisting of an HSP90 inhibitor (e.g. PU-H71, luminespib, ganatespib); an HDAC inhibitor (e.g. panobinostat, givinostat, pracinostat, vorinostat); a DNA methyltransferase inhibitor (e.g. 5-azacytidine, decitabine); an mTOR inhibitor (e.g. rapamycin, everolimus); an AKT inhibitor (e.g. MK-2206); a PI3K inhibitor (e.g. buparlisib, dactolisib); a Hedgehog inhibitor (e.g. glasdegib, saridegib, erismodegib); an SMO inhibitor (e.g. sonidegib, vismodegib); an anti-fibrotic agent, such as simtuzumab, serum amyloid P or a monoclonal antibody (e.g. fresolimumab, simtuzumab); an Aurora-A kinase inhibitor (e.g. dimetylfasudil, alisertib); a TNF-alpha modulator (e.g. danazol); an immunomodulatory agent (e.g. lenalidomide, pomalidomide, thalidomide); a glucocorticoid (e.g. prednisone); a telomerase inhibitor (e.g. imetelstat); an anti-anemic agent (e.g. an erythropoiesis stimulating agent such as sotatercept); a CYP3A4 inhibitor (e.g. ketoconazole, clarithromycin, itraconazole, nefazodone, telithromycin); and a dual CYP2C9-CYP3A4 inhibitor (e.g. fluconazole); or, in each case, a pharmaceutically acceptable salt thereof.

The term “combination” or “pharmaceutical combination” used herein, refers to a non-fixed combination where an active agent and at least one further active agent may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The term “non-fixed combination” means that the active ingredients, e.g. one active agent and at least one further active agent, are both administered to a patient as separate entities either simultaneously or sequentially with no specific time limits, wherein such administration provides therapeutically effective levels of the two compounds in the body of the patient. In particular, reference to crizanlizumab or a binding fragment thereof in combination with ruxolitinib or a pharmaceutically acceptable salt thereof as used herein (e.g. in any of the embodiments or in any of the claims herein), refers to a “non-fixed combination”; and reference to ruxolitinib or a pharmaceutically acceptable salt thereof as used herein (e.g. in any of the embodiments or in any of the claims herein), in combination with at least one further active agent (crizanlizumab being excluded) refers to either a fixed combination in one unit dosage form (e.g., capsule, tablet, caplets or particulates), a non-fixed combination, or a kit-of-parts for the combined administration wherein ruxolitinib or a pharmaceutically acceptable salt thereof and one or more combination partner (e.g. another drug as specified herein, also referred to as further “pharmaceutical active ingredient”, “therapeutic agent” or “co-agent”) may be administered independently at the same time or separately within time intervals.

The term “therapeutically effective amount” refers to an amount of a drug or a therapeutic agent that will elicit the desired biological and/or medical response of a tissue, system or an animal (including man) that is being sought by a researcher or clinician.

### Administration and Treatment Regimen

In one aspect the present invention provides an anti-P-selectin antibody or binding fragment thereof, suitably crizanlizumab or a binding fragment thereof, for use in the treatment of myelofibrosis in a patient, preferably primary myelofibrosis, where the anti-P-selectin antibody or binding fragment thereof, is administered to the patient in a dose between 2.5 mg per kg body weight (2.5 mg/kg) to 20 mg/kg, suitably 2.5 mg/kg to 10 mg/kg in each incidence of administration (dose). Preferably each dose is 5 mg/kg, 7.5 mg/kg or 10 mg/kg. Suitably the dose stays unchanged throughout the treatment. Equally suitably the dose is adjusted according to the disease condition, either up titrated or down titrated.

In one embodiment, the anti-P-selectin antibody or binding fragment thereof, suitably crizanlizumab or a binding fragment thereof, is administered to the patient every 4 weeks (+/−3 days).

In order to quickly exert therapeutic effect or to achieve steady state concentration, it is preferred that the first two doses are provided 2 weeks (+/−3 days) apart followed by further doses provided every 4 weeks (+/−3 days), wherein each dose is between 2.5 mg/kg to 20 mg/kg. Preferably each dose is 5 mg/kg, 7.5 mg/kg or 10 mg/kg.

Suitably the anti-P-selectin antibody or binding fragment thereof, suitably crizanlizumab or a binding fragment thereof, is provided to the subject intravenously.

In one embodiment the present invention provides an anti-P-selectin antibody or binding fragment thereof, suitably crizanlizumab or a binding fragment thereof, for use in the treatment of myelofibrosis, wherein said anti-P-selectin antibody, or binding fragment thereof, is administered in combination with ruxolitinib, or a pharmaceutically acceptable salt thereof. Suitably ruxolitinib is administered in an amount of from 5 mg twice daily to 25 mg twice daily, such as 5 mg twice daily, 10 mg twice daily, 15 mg twice daily, 20 mg twice daily or 25 mg twice daily, depending on the patient's blood count according to the prescribing information for Jakavi®/Jakafi® and the judgment of the treating physician.

All publications and patent applications mentioned in the specification are indicative of the level of skill of those skilled in the art to which the presently disclosed inventive concepts pertain. All publications and patent applications are herein incorporated by reference to the same extent as if each individual publication or patent application was specifically and individually indicated to be incorporated by reference.

The Examples below are set forth to aid in the understanding of the inventions but are not intended to, and should not be construed to, limit its scope in any way.

### EXPERIMENTAL

### 1. Preclinical Studies on the Use of the P-Selectin Inhibitor SEG101 (Crizanlizumab) to Halt Progression of Myelofibrosis in the Gata1^{low} Mouse Model

Experiment 1: In this experiment, murine P-selectin is inhibited with the monoclonal antibody mRB40.34, alone or in combination with ruxolitinib, to assess if treatment reduces the number of thrombotic events in Gata1^{low} mice as they age and to assess if pharmacological inhibition of P-selectin halts the progression of pre-MF to MF in Gata1low mice.

Gata1^{low} mice (5-6-month of age) are divided into five groups (eight mice per group):
Group 1: Vehicle treated (2% v/v DMSO in H2O) (negative control for group 3 and 4)
Group 2: Mice receive the commercially available anti-mouse P-selectin mAb RB40.34 (30 ug/mouse/day), as described (Embury S H et al, Blood 2004; 104:3378-85; Kaul D K et al, J Clin Invest 2000; 106:411-20).
Group 3: Mice receive ruxolitinib alone (45 mg/kg twice per day by gavage in 2% v/v DMSO in H2O), as described (Zingariello M et al, Blood Cancer Journal 2017; 7(6): e572).
Group 4: Mice receive the anti-mouse P-selectin mAb RB40.34p and ruxolitinib in combination
Group 5: Mice receive unfractionated porcine heparin (1.6 U/day/mouse) or anti-mouse E-selectin mAb (10E9.6, Pharmigen) (30 ug/mouse/day) alone (negative controls for group 2).

Mice are treated daily for 5 days (Monday to Friday), allowed to rest for 2 days (Saturday and Sunday) and then treated again for 5 days. These treatments continue for one month. At that point, mice are sacrificed and their liver, spleen, heart and kidney analyzed for signs of thrombotic events by immunohistochemistry with antibodies against fibrinogen. Correlative experiments include flow-cytometric determination of platelet size and cell-surface P-selectin expression, evaluation of bleeding times after tail vein puncture and survival after small surgery. It is expected that pharmacological inhibition of P-selectin prevents thrombus formation in the organs of Gata1^{low} mice. It is expected that these effects are specific for P-selectin inhibition and are not be observed in the heparin/E-selectin and ruxolitinib only group. It is anticipated that the effects of P-selectin inhibition by the anti-P-selectin antibody are enhanced by the addition of ruxolitinib.

Splenomegaly is a major manifestation of PMF contributing to clinical symptoms and hematologic abnormalities. The spleen from PMF patients contains increased numbers of hematopoietic stem cells (HSC) and megakaryocytes. It is hypothesized that megakaryocytes in the MF spleen express high levels of P-selectin, which triggers neutrophil emperipolesis, which leads to disease progression due to the release of TGF-β, a growth factor that has been previously demonstrated to promote the formation of a MF-specific HSC supporting a splenic microenvironment.

Experiment 2: In this experiment, murine P-selectin is inhibited with the monoclonal antibody mRB40.34, alone or in combination with ruxolitinib, to assess if treatment prevents disease progression in Gata1^{low} mice by preventing the development of marrow fibrosis.

It is hypothesized that in the Gata1^{low} mouse model disease progression is sustained by a P-selectin/TGF-β circuit. It is proposed that in Gata1^{low} mice, hematopoiesis in the spleen is sustained by a circuit between P-selectin and TGF-β and contributes to disease progression. This circuit is triggered by the abnormal expression of P-selectin on MK that leads to neutrophil-MK emperipolesis, increasing TGF-β content and resulting in fibrocyte activation. Activated fibrocytes establish, possibly through P-selectin, peripolesis with MK forming “myelofibrosis-related stem cell niches” that sustain proliferation of these cells in spleen generating more MK and more neutrophils, establishing an amplification loop that contributes to disease progression. This loop may also determine hematopoietic failure and fibrosis in BM.

Given the well described effects of inhibition of the TGF-β receptor 1 kinase on myelofibrosis in mouse models, the effects of the P-selectin inhibitor with those obtained with SB431542 are compared. In fact, contrary to inhibition of TGF-β signaling, inhibition of P-selectin has limited side effects and is therefore preferable to TGF-β inhibitors. Parallel experiments are performed with SB431542 alone or in combination with ruxolitinib. Gata1^{low} mice are treated at 5-6 months of age (i.e. in the pre-MF stage) and analyzed at 10-12 months, an age when they are expected to have MF.

### Experiment 2a: P-Selectin Inhibition

Gata1^{low} mice (5-6-month of age) are divided into five groups (eight mice per group):
Group 1: Vehicle treated (2% v/v DMSO in H2O) (negative control for group 3 and 4)
Group 2: Mice receive the commercially available anti-mouse P-selectin mAb RB40.34 (30 ug/mouse/day)
Group 3: Mice receive ruxolitinib alone (45 mg/Kg twice per day by gavage in 2% v/v DMSO in H2O)
Group 4: Mice receive the anti-mouse P-selectin mAb RB40.34p and ruxolitinib in combination
Group 5: Mice receive unfractionated porcine heparin (1.6 U/day/mouse) or anti-mouse E-selectin mAb (10E9.6, Pharmigen) (30 ug/mouse/day) alone (negative controls for group 2).

### Experiment 2b: TGF-β Receptor 1 Kinase Inhibition

Gata1^{low} mice (8 mice per group) are treated with SB431542 according to the following scheme:
Group 1: Vehicle treated (2% v/v DMSO in H2O) (negative control)
Group 2: Mice receive SB431542 (60 μg/kg/day, cat no S4317-5GM, Sigma-Aldrich, St Louis, MO), as described (Spangrude G J et al, Stem Cells 2016; 34:67-82; Zingariello M et al, Blood 2013; 121:3345-63).
Group 3: Mice receive ruxolitinib alone (45 mg/kg twice per day by gavage in 2% v/v DMSO in H2O)
Group 4: Mice receive SB431542 and ruxolitinib in combination

5-6 months old mice are treated daily for 5 days (Monday to Friday), then rested for 2 days and then treated again for 5 days. These treatments continue until the mice will reach 10-12 months of age. At that point they are sacrificed and analyzed for signs of progression to MF as described by Spangrude G J et al, Stem Cells 2016; 34:67-82; Zingariello M et al, Blood 2013; 121:3345-63.

End-points of this study include blood counts and histopathological examination for fibrosis, neoangiogenesis, osteosclerosis and hematopoiesis in marrow and spleen.

It is expected that pharmacological inhibition of P-selectin recapitulate the results obtained with genetic deletion and halts progression of MF in Gata1^{low} mice. It is expected that these effects are specific for P-selectin inhibition and are not observed in the heparin/E-selectin group and that the effect are enhanced by ruxolitinib. It is expected that the results of treatment with SB431542 are similar to those obtained with the P-selectin inhibitor but that SB431542 is associated with a poor toxicity profile (increased osteosclerosis).

### 2. Clinical Testing

Clinical testing of crizanlizumab, alone or in combination with ruxolitinib, are conducted, for example, according to standard clinical practice (e.g. placebo control study, for example in analogy to COMFORT-1 trial) in patients with myelofibrosis, in particular with primary myelofibrosis.

Key inclusion criteria include diagnosis of PMF, PPV-MF or PET-MF, in men or women, aged 18 years or older, with palpable spleen length of 5 cm or greater measuring below the costal margin, who are classified as high risk (3 or more prognostic factors) OR intermediate risk level 2 (2 prognostic factors) defined by the International Working Group (Cervantes et al, Blood 2009 113:2895-2901), and for whom treatment of MF is indicated based on one or more of the following indications: (1) classification as high risk by the Cervantes et al, 2009 criteria; (2) palpable splenomegaly of 10 cm or greater below the costal margin or (3) active symptoms of MF as designated by protocol defined scores on the Screening Symptom Form. Subjects must have peripheral blast count <10%, have absolute CD34+ cell count >20×10⁶/L and be naïve to JAK inhibitor therapy. Subjects must be refractory, resistant or intolerant to available therapy, or, in the investigator's judgment, are not candidates for available therapy.

### Primary Efficacy Endpoint:

Proportion of subjects achieving ≥35% reduction in spleen volume from Baseline to Week 24 as measured by MRI (or CT scan in applicable subjects).

### Safety and Tolerability:

Safety and tolerability will be assessed by monitoring the frequency, duration and severity of adverse events, performing physical exams, and evaluating changes in vital signs, electrocardiograms (ECGs), serum chemistry, hematology and urinalysis results

### Secondary Efficacy Endpoints:

Duration of maintenance of a ≥35% reduction from Baseline in spleen volume among subjects initially randomized to receive 1) crizanlizumab or 2) crizanlizumab and ruxolitinib.
·Proportion of subjects who have ≥50% reduction in total symptom score from Baseline to Week 24 as measured by the Modified MFSAF v2.0 diary.
Change in total symptom score from Baseline to Week 24 as measured by the modified MFSAF v2.0 diary.
Overall survival.

1. A method of treating myelofibrosis (MF) in a patient comprising administering a therapeutically effective amount of an anti-P-selectin antibody or binding fragment thereof.

2. The method of claim 1, wherein myelofibrosis comprises primary myelofibrosis (PMF), post-essential thrombocythemia myelofibrosis (PET-MF) and post-polycythemia vera myelofibrosis (PPV-MF).

3. The method of claim 1, wherein myelofibrosis is primary myelofibrosis (PMF).

4. The method of claim 1,
wherein median survival time increases by at least 3 months.

5. The method of claim 1,
wherein said patient completely responds to the treatment.

6. The method of claim 1, wherein said MF is newly diagnosed MF.

7. The method of claim 1, wherein said P-selectin antibody, or binding fragment thereof, is administered in combination with at least one further active agent.

8. The method of claim 7, wherein the at least one further active agent is a JAK1/JAK2 inhibitor, a JAK2/FLT3 inhibitor, a JAK2V617F inhibitor, a JAK2 inhibitor, JAK1 inhibitor or a JAK2/Src inhibitor, such as ruxolitinib, or a pharmaceutically acceptable salt thereof.

9. The method of claim 8, wherein ruxolitinib or a pharmaceutically acceptable salt thereof, is administered in an amount of from 5 mg twice daily to 25 mg twice daily, such as 5 mg twice daily, 10 mg twice daily, 15 mg twice daily, 20 mg twice daily or 25 mg twice daily.

10. The method of claim 1, wherein said P-selectin antibody or a binding fragment thereof, is crizanlizumab or a binding fragment thereof.

11. The method of claim 10, wherein crizanlizumab is administered in an amount of from 2.5 mg/kg to 20 mg/kg, in particular in an amount of 5 mg/kg or 7.5 mg/kg.

12. The method of claim 10, wherein crizanlizumab is administered every 4 weeks (+/−3 days).

13. The claim 11, wherein the first two doses of crizanlizumab are provided 2 weeks (+/−3 days) apart followed by further doses provided every 4 weeks (+/−3 days), wherein each dose is between 2.5 mg/kg to 20 mg/kg.

14. The method of claim 13, wherein each dose of crizanlizumab is administered in an amount of 5 mg/kg or 7.5 mg/kg.

15. The method of claim 7, wherein the at least one further active agent is selected from the group consisting of an HSP90 inhibitor (e.g. PU-H71, luminespib, ganatespib); an HDAC inhibitor (e.g. panobinostat, givinostat, pracinostat, vorinostat); a DNA methyltransferase inhibitor (e.g. 5-azacytidine, decitabine); an mTOR inhibitor (e.g. rapamycin, everolimus); an AKT inhibitor (e.g. MK-2206); a PI3K inhibitor (e.g. buparlisib, dactolisib); a Hedgehog inhibitor (e.g. glasdegib, saridegib, erismodegib); an SMO inhibitor (e.g. sonidegib, vismodegib); an anti-fibrotic agent, such as simtuzumab, serum amyloid P or a monoclonal antibody (e.g. fresolimumab, simtuzumab); an Aurora-A kinase inhibitor (e.g. dimetylfasudil, alisertib); a TNF-alpha modulator (e.g. danazol); an immunomodulatory agent (e.g. lenalidomide, pomalidomide, thalidomide); a glucocorticoid (e.g. prednisone); a telomerase inhibitor (e.g. imetelstat); an anti-anemic agent (e.g. an erythropoiesis stimulating agent such as sotatercept); a CYP3A4 inhibitor (e.g. ketoconazole, clarithromycin, itraconazole, nefazodone, telithromycin); and a dual CYP2C9-CYP3A4 inhibitor (e.g. fluconazole); or, in each case, a pharmaceutically acceptable salt thereof.

16. The method of claim 7, wherein the at least one further active agent is ruxolitinib or a pharmaceutically acceptable salt thereof and wherein at least one additional further active agent is selected from the group consisting of an HDAC inhibitor (e.g. panobinostat, givinostat, pracinostat, vorinostat); a DNA methyltransferase inhibitor (e.g. 5-azacytidine, decitabine); an mTOR inhibitor (e.g. rapamycin, everolimus); an AKT inhibitor (e.g. MK-2206); a PI3K inhibitor (e.g. buparlisib, dactolisib); a Hedgehog inhibitor (e.g. glasdegib, saridegib, erismodegib); an SMO inhibitor (e.g. sonidegib, vismodegib); an anti-fibrotic agent, such as simtuzumab, serum amyloid P or a monoclonal antibody (e.g. fresolimumab, simtuzumab); an Aurora-A kinase inhibitor (e.g. dimetylfasudil, alisertib); a TNF-alpha modulator (e.g. danazol); an immunomodulatory agent (e.g. lenalidomide, pomalidomide, thalidomide); a glucocorticoid (e.g. prednisone); a telomerase inhibitor (e.g. imetelstat); an anti-anemics agent (e.g. an erythropoiesis stimulating agent such as sotatercept); a CYP3A4 inhibitor (e.g. ketoconazole, clarithromycin, itraconazole, nefazodone, telithromycin); and a dual CYP2C9-CYP3A4 inhibitor (e.g. fluconazole); or, in each case, a pharmaceutically acceptable salt thereof.
